(19) United States
(12) Patent Application Publication
(10) Pub. No.: US 2007/0185388 A1
(43) Pub. Date: Aug. 9, 2007

(54) Thorax mounted stabilization platform

(75) Inventors: Arthur A. Bertolero, Danville, CA (US); Tamer Ibrahim, Pleasant Hill, CA (US)

Correspondence Address: GREGORY SMITH & ASSOCIATES, 3900 NEWPARK MALL ROAD, 3RD FLOOR, NEWARK, CA, 94560, US

(73) Assignee: ENDOSCOPIC TECHNOLOGIES, INC.

(21) Appl. No.: 11/729,326

(22) Filed: Mar. 27, 2007

Related U.S. Application Data
(63) Continuation of application No. 10/988,027, filed on Nov. 12, 2004, now abandoned.
(60) Provisional application No. 60/519,221, filed on Nov. 11, 2003.

(51) Int. Cl. A61B 1/32

(52) U.S. Cl. 600/234; 600/228

(57) ABSTRACT

A thorax mounted stabilizing platform for a surgical device, such as a tissue stabilizer, can be inserted through a minimally invasive incision and affixed to the thoracic wall to stabilize the surgical device. The stabilizing platform includes a rod that is introduced into a percutaneous opening in the patient. An internal and/or an external fixing device is deployed to attach the rod to the patient. One or more surgical devices may be mounted to the distal or internal end of the rod. An adjustment knob or other actuation mechanism is located at the proximal or external end of the rod to actuate or manipulate the surgical device(s) attached to the distal end.

### CROSS REFERENCE TO RELATED APPLICATIONS

This is a continuation of U.S. Utility Application No. 10/988,027, filed Nov. 12, 2004, now abandoned, which claimed the benefit of U.S. Provisional Application No. 60/519,221, filed on Nov. 11, 2003. This and all patents and patent applications referred to herein are hereby incorporated by reference in their entirety.

### FIELD OF THE INVENTION

The invention pertains to apparatus and methods for delivery and use of surgical devices. In particular, it is a stabilization platform mounted on the thorax of the patient for use with endoscopic surgical tools.

### BACKGROUND OF THE INVENTION

Surgery on the heart is one of the most commonly performed types of surgery that is done in hospitals across the U.S. Cardiac surgery can involve the correction of defects in the valves of the heart, defects to the veins or the arteries of the heart and defects such as aneurysms and thromboses that relate to the circulation of blood from the heart to the body. Coronary artery bypass graft (CABG) surgery is one of the most common cardiac surgery procedures. In the past, most cardiac surgery was performed as open-chest surgery, in which a primary median sternotomy was performed. That procedure involves vertical midline skin incision from just below the super sternal notch to a point one to three centimeters below the tip of the xiphoid. This is followed by scoring the sternum with a cautery, then dividing the sternum down the midline and spreading the sternal edges to expose the area of the heart in the thoracic cavity. This technique causes significant physical trauma to the patient and can require one week of hospital recovery time and up to eight weeks of convalescence. This can be very expensive in terms of hospital costs and disability, to say nothing of the pain to the patient.

Recently, attempts have been made to change such invasive surgery to minimize the trauma to the patient, to allow the patient to recover more rapidly and to minimize the cost involved in the process. New surgical techniques have been developed which are less invasive and traumatic than the standard open-chest surgery. This is generally referred to as minimally-invasive surgery. One of the key aspects of the minimally invasive techniques is the use of a trocar cannula as an entry port for the surgical instruments. In general, minimally invasive surgery entails several steps: (1) at least one, and preferably at least two, intercostal incisions are made to provide an entry position for a trocar; (2) a trocar is inserted through the incision to provide an access channel to the region in which the surgery is to take place, e.g., the thoracic cavity; (3) a videoscope is provided through another access port to image the internal region (e.g., the heart) to be operated on; (4) an instrument is inserted through the trocar channel, and (5) the surgeon performs the indicated surgery using the instruments inserted through the access channel. Prior to steps (1)-(5), the patient may be prepared for surgery by placing him or her on a cardiopulmonary bypass (CPB) system and the appropriate anesthesia, then maintaining the CPB and anesthesia throughout the operation. See U.S. Pat. No. 5,452,733 to Sterman et al. issued Sep. 26, 1995 for a discussion of this technique.

While this procedure has the advantage of being less invasive or traumatic than performing a media, sternotomy, there are numerous disadvantages to using trocars to establish the entry ports for the instruments and viewscope. For example, the trocars are basically “screwed” into position through the intercostal incision. This traumatizes the local tissues and nerve cells surrounding the trocar.

Once in place, the trocar provides a narrow cylindrical channel having a relatively small circular cross-section. This minimizes the movement of the instrument relative to the longitudinal axis and requires specially-designed instruments for the surgeon to perform the desired operation (See, e.g., the Sterman patent U.S. Pat. No. 5,452,733). In addition, because of the limited movement, the surgeon often has to force the instrument into an angle that moves the trocar and further damages the surrounding tissue and nerves. The need to force the instrument causes the surgeon to lose sensitivity and tactile feedback, thus making the surgery more difficult. The surgical retractor of this invention is designed to reduce the initial trauma to the patient in providing access to the internal region, to reduce the trauma to the patient during surgery, to provide the surgeon with greater sensitivity and tactile feedback during surgery, and to allow the surgeon to use instruments of a more standard design in performing the non-invasive surgery.

Other less invasive surgical techniques include access to the region of the heart to be corrected by anterior mediastinotomy or a thoracotomy. In a mediastinotomy, a parasternal incision is made that is two to three inches in length on the left or the right of the patient's sternum according to the cardiac structure that needs the attention in the surgery. Either the third or the fourth costal cartilage is excised depending on the size of the heart. This provides a smaller area of surgical access to the heart that is generally less traumatic to the patient. A thoracotomy is generally begun with an incision in the fourth or fifth intercostal space, i.e. the space between ribs 4 and 5 or ribs 5 and 6. Once an incision is made, it is completed to lay open underlying area by spreading the ribs. A retractor is used to enlarge the space between the ribs.

At the present time, when either of these techniques are used, a retractor is used to keep the ribs and soft tissues apart and expose the area to be operated on to the surgeon who is then able to work in the surgical field to perform the operation. The types of retractors that are used may be seen, for example, in volume 1 of Cardiac Surgery by John W. Kirkland and Brian G. Barratt-Boyes, Second Edition, Chapter 2, at page 101. Commercial-type retractors for minimally-invasive surgery that are useful for a mediastinotomy or a thoracotomy are manufactured by Snowden Pencer (the ENDOCABG rib spreader and retractor), U.S. Surgical (the mini CABG system), and Cardiothoracic Systems (the CTS MIDCAB. System). The ENDOCABG refractor is two opposing retractor arms that are interconnected by a ratchet arm having a thumbscrew which can adjust the distance between the retractor arms. While this provides a useful retractor, it has certain shortcomings in its ease of use. The mini CABG System is an oval-based platform to which a number of retractors are then fitted around the extremity of the universal ring base and adjusted by a gear tooth connection. Each of the retractors have to be separately adjusted and there are other devices that can be connected to the universal base which can aid the surgeon in damping the heart movement to better work on the artery or vessel to which the surgeon is directing his attention. The CTS MIDCAB. System serves a similar function to the ENDOCABG retractor, but is more complex.

Off-pump coronary artery bypass (OPCAB) surgery is a variation of the CABG procedure that is performed on a patient's beating heart. OPCAB surgery can be performed using minimally invasive techniques or using a sternotomy or other thoracotomy for surgical access. A tissue stabilizer is often used for stabilizing an area of tissue on the patient's beating heart to facilitate an anastomosis between the graft vessel and the coronary artery. Examples of tissue stabilizers for OPCAB surgery are described in PCT International Patent Application WO 01/58362 Tissue stabilizer and in U.S. Pat. No. 6,755,780 Method and apparatus for temporarily immobilizing a local area of tissue. Such tissue stabilizers are typically mounted to the surgical retractor or to the surgical table to provide a stable platform for immobilizing the area of tissue. A disadvantage of this approach is that the tissue stabilizer tends to crowd the surgical field, which is particularly a problem when using small minimally invasive incisions for performing the surgery. It would be desirable therefore to provide a stabilizing platform for a surgical device, such as a tissue stabilizer, that can be inserted through a separate minimally invasive incisions and that does not need to be mounted on the surgical retractor or the surgical table for stability.

### SUMMARY OF THE INVENTION

In keeping with the foregoing discussion, the present invention provides a thorax mounted stabilizing platform for a surgical device, such as a tissue stabilizer, that can be inserted through a separate minimally invasive incision and that does not need to be mounted on the surgical retractor or the surgical table for stability. The stabilizing platform can be affixed to the thorax of a patient during a surgical procedure. A rod is introduced into a percutaneous opening in the patient. An internal and/or an external fixing device is deployed to attach the rod to the patient. One or more surgical devices may be mounted to the distal or internal end of the rod. An adjustment knob or other actuation mechanism is located at the proximal or external end of the rod to actuate or manipulate the surgical device(s) attached to the distal end.

### BRIEF DESCRIPTION OF THE DRAWINGS

FIG. 1 shows a first embodiment of the stabilization platform with an external stabilizer affixed to an exterior surface the patient's thorax.

FIG. 2 shows a second embodiment of the stabilization platform with an internal stabilizer affixed to an interior surface the patient's thoracic cavity.

FIG. 3 shows a third embodiment of the stabilization platform with an internal stabilizer and an external stabilizer.

FIGS. 4 and 5 show a fourth embodiment of the stabilization device with an external stabilizer and a remotely actuatable internal stabilizer.

FIG. 6 shows a fifth embodiment of the stabilization platform with an inflatable internal stabilizer and an inflatable external stabilizer.

### DETAILED DESCRIPTION

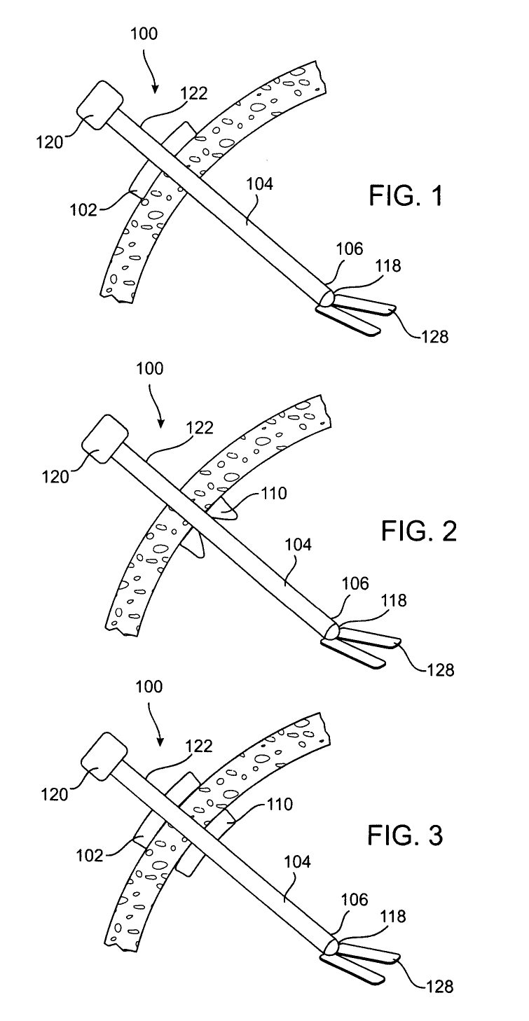

FIG. 1 shows a first embodiment of the stabilization platform 100. In this embodiment, an external stabilizer 102 is used to hold the platform in place during the surgical procedure being performed. The external stabilizer 102 is an object located around or attached to a rod 104 that prevents the distal end 106 of the rod from extending too far into the patient. The external stabilizer 102 may be located at a fixed point on the rod 104. Alternatively, the user may select a depth to which the end of the surgical instrument or rod 104 should extend and then move the external stabilizer 102 to the appropriate location along the rod 104. The rod 104 may have depth markers to assist in gauging the appropriate depth. Alternatively, the user may guide the distal end 106 of the rod 104 and/or surgical tool into place by feel or using a known imaging system. Then, the user would slide or place the external stabilizer 102 and affix it to the selected location on the rod 104. To further secure the external stabilizer 102, a medical grade adhesive could be used to affix the external stabilizer 102 to the skin of the patient.

FIG. 2 shows a second embodiment of the stabilization platform. In this embodiment, an internal stabilizer 1 10 is used to hold the device in place during the surgical procedure being performed. The internal stabilizer 110 is an object located around or attached to the rod 104 that prevents the device from being inadvertently removed from the patient or may be used to seal the opening through the wall of the cavity in the patient.

The internal stabilizer 110 may take the form of an elongated member. The narrow direction of the internal stabilizer is sized to fit between the ribs of the patient. Once the internal stabilizer 110 is inserted into the patient, the internal stabilizer is rotated 90 degrees. The long direction of the internal stabilizer 110 is sized such that, after rotation, the ends of the elongated member 110 rest against the internal surface of the thoracic cavity.

In another version, the internal stabilizer is inflatable. Once the internal stabilizer has passed through the opening in the skin and between the ribs, the internal stabilizer is inflated. After inflation, the internal stabilizer exceeds the size of the opening, thereby holding the device in place.

Another embodiment the internal stabilizer deploys mechanically. In this version, one or more projections lie flat against the rod during insertion. After insertion, the projections are extended using a trigger or actuator on the proximal end of the rod. This may be accomplished by many known means including, but not limited to, a spring biased release mechanism, a mechanical interlock, scissor linkages and hinges, etc. If used for sealing the percutaneous opening, an elastic or compressible material may be added to help seal the opening.

To further secure the internal stabilizer, a medical grade adhesive could be used to affix the internal stabilizer to the internal cavity of the patient.

FIG. 3 shows a third embodiment of the stabilization device 100. In this embodiment, both an external stabilizer 102, as described in FIG. 1, and an internal stabilizer 110, as described in FIG. 2, are used to hold the device in place during the surgical procedure being performed. With the use of both the internal and external stabilizers, the device is locked into place and cannot penetrate farther into the patient or move back out of the patient. In this version, the internal stabilizer and external stabilizer may be formed of one or more projections, which act as a clamp and may be selectively placed around a stable structure in the patient, such as a rib. If desired, two pair of projections may be used. In this case, the two pair can clamp onto two adjacent ribs. Additional pairs of projections may be used to further secure the device.

Alternate versions of the above embodiments may be configured with internal or external clamps to attach to other surgical tools, such as the retractor used to widen the incision between ribs.

The rod may be solid or hollow and may be formed of a rigid material such as a stainless steel or plastic tube. Alternately, the rod may be formed of a malleable material that would allow the user to bend the rod into a selected shape prior to insertion. Also, the rod may be fixedly and/or steerably articulated, to allow the user to move the rod into a particular configuration before, during or after insertion into the patient. Once in the selected configuration, the joints may be tightened to make the rod generally rigid.

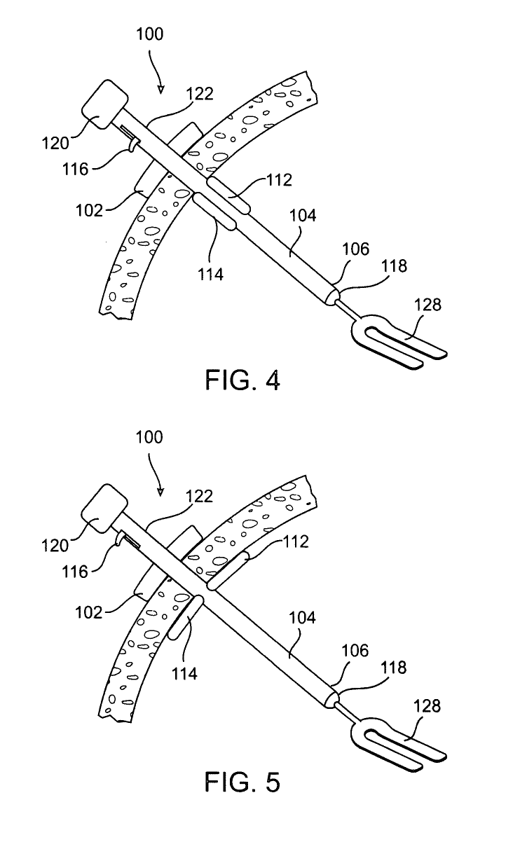

FIGS. 4 and 5 show a fourth embodiment of the stabilization device 100 with an external stabilizer 102 and a remotely actuatable internal stabilizer 110. In this embodiment, the internal stabilizer 110 includes a plurality of stabilizer members 112, 114 pivotally connected to the rod or elongate member 104 and a trigger or other actuator 116 positioned on the device external to the patient's thorax for pivoting the stabilizer members 112, 114 relative to the elongate member 104. The stabilizer members 112, 114 are pivoted to a retracted position for insertion through an incision in the intercostal space and into the patient's thoracic cavity, as shown in FIG. 4. Then, the trigger 116 is actuated the to rotate the stabilizer members 112, 114 from the retracted position to an extended position to contact the interior surface of the patient's thoracic cavity, as shown in FIG. 5.

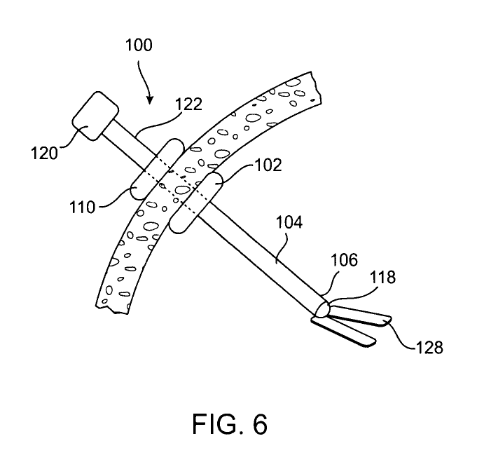

FIG. 6 shows a fifth embodiment of the stabilization platform with an inflatable internal stabilizer 110 and an inflatable external stabilizer 102.

In each embodiment of the thorax mounted stabilization platform 100, the external and/or internal stabilizers 102, 110 can be connected to the elongate member 104 at a fixed location or at an adjustable location between the proximal and distal ends of the elongate member 104. In the latter case, the device will include means for selectively locking the external and/or internal stabilizers 102, 110 at a selected location between the proximal and distal ends of the elongate member 104. In addition, the elongate member 104 may be pivotally connected to the external and/or internal stabilizers 102, 110 and the device may include means for selectively locking the pivotal connection between the elongate 104 member and the external and/or internal stabilizers 102, 110.

The distal end 106 of the rod 104 in each of the embodiments may include a connector 118 for attaching a surgical tool 128. The connector 118 may take the form of a swivel, a ball and socket joint, a ball and collet joint, a hinge, interlock or other fixed or adjustable connector. The connector may be configured to have a snap-in feature such that the ball is held securely by the collet upon insertion, but still allows articulation of ball until the arm is tightened. The frictional characteristics and geometries may also be optimized, such that the connector loses frictional stability at approximately same point at which the remainder of the flexible arm in its tightened state or the strain point of a malleable rod is exceeded.

Control cables, wires, rods or other actuation and/or control mechanisms may run through one or more openings within the rod or up the sides of the rods. If the actuation mechanism is located outside the rod, tubes or channels may be used to prevent interference in operation of the actuation mechanism. The actuation mechanism may be used for several purposes, including, but not limited to positioning of the surgical or medical tool within the patient, control or actuation of the surgical or medical tool, deployment and/or control of the internal stabilizer, remote coupling and uncoupling of a surgical or medical tool.

The actuation mechanism may also include a trigger, actuator, adjustment knob, button, handle, toggle, ratchet, or other known interface 120 on the proximal end 122 of the rod 104, thereby allowing the user to control the surgical or medical tool 128 remotely. This mechanism may be actuated manually, or pneumatically, electronically or with other means of mechanical advantage.

It may also be desirable to utilize a tool that has been inserted into the patient from a remote incision. Therefore, the actuation mechanism may include an actuator for the connector, thereby allowing the user to connect and disconnect a tool to the distal end of the rod while the rod is located within the patient.

Alternately, a collapsible tool may be attached to the distal end of the rod. Using a collapsible tool reduces the size and/or profile of the device during entry through the percutaneous opening, and expands to tool into an operational configuration inside of the chest or other bodily cavity. In this case, the actuation mechanism may include a button, switch or other mechanism on the proximal handle for deploying the collapsible tool. The collapsing mechanism may include, but is not limited to one or more hinge joints, one or more spring-loaded joints, inflation lumen and/or a trigger and locking mechanism.

The stabilization platform may be used with a plurality of surgical or medical tools, including but not limited to heart or other organ stabilizing devices, heart or other organ positioning devices, cutting devices, biopsy devices, injection devices, ablation devices therapeutic agents and devices and diagnostic devices. In a particularly preferred embodiment, the surgical tool 128 attached to the rod 104 of the stabilization platform 100 is a tissue stabilizer for stabilizing an area of tissue on the patient's beating heart for performing an off-pump coronary artery bypass (OPCAB) surgery. Depending on the type of tool being connected, deployed and/or actuated by the stabilizing platform, the tool may be introduced into the cavity through an outer sheath into which the stabilization platform is located or a separate sheath entering the cavity through a different opening.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. A thorax mounted stabilization platform comprising:
an elongate member having a proximal end and a distal end, and configured to extend from an external location through an incision in the patient's thorax and into the patient's thoracic cavity; and
a stabilizer connected to the elongate member and having means for affixing to the patient's thorax.

2. The thorax mounted stabilization platform of claim 1, wherein the stabilizer is connected to the elongate member at a fixed location between the proximal end and the distal end of the elongate member.

3. The thorax mounted stabilization platform of claim 1, wherein the stabilizer is connected to the elongate member at an adjustable location between the proximal end and the distal end of the elongate member.

4. The thorax mounted stabilization platform of claim 3, further comprising means for selectively locking the stabilizer at a selected location between the proximal end and the distal end of the elongate member.

5. The thorax mounted stabilization platform of claim 1, wherein the elongate member is pivotally connected to the stabilizer.

6. The thorax mounted stabilization platform of claim 5, further comprising means for selectively locking the pivotal connection between the elongate member and the stabilizer.

7. The thorax mounted stabilization platform of claim 1, further comprising a surgical device or instrument connected to the distal end of the elongate member.

8. The thorax mounted stabilization platform of claim 7, wherein the surgical device or instrument comprises a tissue stabilizer.

9. The thorax mounted stabilization platform of claim 7, further comprising means for adjusting a position of the surgical device or instrument relative to the elongate member from a position external to the patient's thorax.

10. The thorax mounted stabilization platform of claim 1, wherein the distal end of the elongate member is adapted for removably attaching a surgical device or instrument to the elongate member.

11. The thorax mounted stabilization platform of claim 10, wherein the surgical device or instrument comprises a tissue stabilizer.

12. The thorax mounted stabilization platform of claim 10, further comprising means for selectively grasping or releasing of the surgical device or instrument at the distal end of the elongate member from a position external to the patient's thorax.

13. The thorax mounted stabilization platform of claim 10, further comprising means for adjusting a position of the surgical device or instrument relative to the elongate member from a position external to the patient's thorax.

14. The thorax mounted stabilization platform of claim 1, wherein the stabilizer is pivotally connected to the elongate member at an adjustable location between the proximal end and the distal end of the elongate member, and further comprising means for selectively locking the pivotal connection between the elongate member and the stabilizer and for locking the stabilizer at a selected location between the proximal end and the distal end of the elongate member.

15. The thorax mounted stabilization platform of claim 1, wherein the stabilizer is an internal stabilizer having means for affixing to an interior surface of the patient's thoracic cavity.

16. The thorax mounted stabilization platform of claim 15, wherein the means for affixing to an interior surface of the patient's thoracic cavity comprises a medical grade adhesive.

17. The thorax mounted stabilization platform of claim 15, wherein the internal stabilizer comprises at least one inflatable member.

18. The thorax mounted stabilization platform of claim 15, wherein the internal stabilizer comprises at least one stabilizer member pivotally connected to the elongate member.

19. The thorax mounted stabilization platform of claim 15, wherein the internal stabilizer comprises a plurality of stabilizer members pivotally connected to the elongate member and an actuator for pivoting the stabilizer members relative to the elongate member from a position external to the patient's thorax.

20. The thorax mounted stabilization platform of claim 1, wherein the stabilizer is an external stabilizer having means for affixing to an exterior surface of the patient's thorax.

21. The thorax mounted stabilization platform of claim 20, wherein the means for affixing to an exterior surface of the patient's thorax comprises a medical grade adhesive.

22. The thorax mounted stabilization platform of claim 20, wherein the external stabilizer comprises at least one inflatable member.

23. The thorax mounted stabilization platform of claim 20, wherein the external stabilizer comprises at least one stabilizer member pivotally connected to the elongate member.

24. The thorax mounted stabilization platform of claim 1, wherein the stabilizer comprises an external stabilizer configured to contact an exterior surface of the patient's thorax and an internal stabilizer configured to contact an interior surface of the patient's thoracic cavity.

25. The thorax mounted stabilization platform of claim 24, wherein the internal stabilizer comprises a plurality of stabilizer members pivotally connected to the elongate member and an actuator for pivoting the stabilizer members relative to the elongate member from a position external to the patient's thorax.

26. The thorax mounted stabilization platform of claim 1, wherein the stabilizer is pivotally connected to the elongate member at an adjustable location between the proximal end and the distal end of the elongate member, and further comprising means for selectively locking the pivotal connection between the elongate member and the stabilizer and for locking the stabilizer at a selected location between the proximal end and the distal end of the elongate member, wherein the stabilizer comprises an external stabilizer configured to contact an exterior surface of the patient's thorax and an internal stabilizer configured to contact an interior surface of the patient's thoracic cavity, and wherein the internal stabilizer comprises a plurality of stabilizer members pivotally connected to the elongate member and an actuator for pivoting the stabilizer members relative to the elongate member from a position external to the patient's thorax.

27. The thorax mounted stabilization platform of claim 26, wherein the distal end of the elongate member is adapted for removably attaching a surgical device or instrument to the elongate member, and further comprising means for selectively grasping or releasing of the surgical device or instrument at the distal end of the elongate member from a position external to the patient's thorax.
